(19) United States
(12) Patent Application Publication
(10) Pub. No.: US 2008/0247561 A1
(43) Pub. Date: Oct. 9, 2008

(54) VARIABLE FIT EAR INSERT

(76) Inventor: Richard C. Smith, Costa Mesa, CA (US)

Correspondence Address: MACPHERSON KWOK CHEN & HEID LLP, 2033 GATEWAY PLACE, SUITE 400, SAN JOSE, CA, 95110, US

(21) Appl. No.: 11/869,517

(22) Filed: Oct. 9, 2007

Related U.S. Application Data
(63) Continuation-in-part of application No. 11/411,314, filed on Apr. 26, 2006.
Application No. 11/411,314 is a continuation-in-part of application No. 11/247,105, filed on Oct. 11, 2005.

(51) Int. Cl. A61F 11/06

(52) U.S. Cl. 381/72

(57) ABSTRACT

An ear insert for protecting the ear from loud noise can have a stem that is configured to be inserted into an ear canal. One or more attachments can be configured to attach to the stem in a manner that at least partially defines a fit of the ear insert within an ear canal. For example, rubber o-rings can be can be selected according to their size so as to provide a desirable fit of the insert within the ear canal. In this manner, the undesirable leakage of ambient noise into the ear canal is mitigated and hearing protection is enhanced.

### RELATED APPLICATIONS

This patent application is a continuation-in-part (CIP) patent application of U.S. patent application Ser. No. 11/411,314, filed on Apr. 26, 2006, and entitled EARPIECE WITH EXTENSION (docket no. M-15744-1P US), which is a continuation-in-part (CIP) patent application of U.S. patent application Ser. No. 11/247,105, filed on Oct. 11, 2005, and entitled EARPIECE WITH FLANGED EXENSION (docket no. M-15744 US), the entire contents of both of which are hereby expressly incorporated by reference.

### TECHNICAL FIELD

The present invention relates generally to hearing protection. The present invention relates more particularly to an insert for an earpiece, wherein the fit of the insert can be varied so as to accommodate a range of ear canal sizes and configurations.

### BACKGROUND

It is well known that high levels of ambient noise can cause hearing loss. Hearing protection can be used in noisy environments in an attempt to mitigate hearing loss. Hearing protection is commonly used in such noisy environments as manufacturing facilities, warehouses, construction sites, shooting ranges, battlefields, and airports. Typically, any time that loud machinery is being operated, guns are being fired, or any other source of excessive noise is present, hearing protection is desirable so as to reduce discomfort and so as to conserve hearing. Hearing protection can lower the level of sound substantially before it reaches the eardrum. Thus, hearing protection can mitigate damage to the ear.

Contemporary hearing protection typically comprises either a noise reducing headset or earplugs. Headsets have cups or earpieces that generally surround the outer ear and reduce the level of noise that is incident thereon. Earplugs, by way of contrast, are inserted into the ear canal, where they reduce the level of sound transmitted therethrough to the eardrum. Both headsets and earplugs have proven effective in conserving hearing.

Although contemporary headsets are generally effective in reducing noise exposure, they suffer from deficiencies that tend to make them unsuitable for some applications. Headsets are bulky. They cannot be worn in environments where they can get in the way, and they cannot be worn under helmets. Headsets are subject to being knocked off of a person's head by others and by items in the user's environment.

Headsets often become uncomfortable after being worn for an extended period of time. Because of this, users can discontinue their use, i.e., remove the headset, while they remain within a noisy environment. Users can alternatively stretch the earpieces of the headset apart from one another, so that they do not apply as much pressure to the sides of the head. This may be done in an attempt to decrease discomfort. However, when the pressure is reduced in this manner, then the cups of the headset may not seal properly and thus may permit the introduction of excessive sound. Noise exposure is undesirably increased and the likelihood of hearing loss is consequently increased as well.

Contemporary earplugs can also be effective. Earplugs can generally be worn with helmets. Earplugs are not as susceptible to being knocked off of a user as are headsets. However, like headsets, earplugs can become uncomfortable when worn for extended periods of time. This is particularly true if the earplugs do not fit properly.

Further, earplugs frequently tend to work loose during usage, thus reducing their effectiveness. They may even fall out. Again, this is particularly true if the earplugs do not fit properly. Of course, when they fall out, the earplugs provide no protection at all. When earplugs fall out, they may be lost so that they cannot subsequently be re-inserted.

Earpieces for use with two-way radios are well known. Law enforcement, security, and military personnel frequently wear earpieces that fit into the conchae bowl of the ear so as to facilitate listening to two-way radios and the like. Such earpieces are different from earplugs in that they are intended to allow some sound to enter the ear. These earpieces are attached via acoustic tubing to a small speaker. Communications received by a two-way radio are reproduced by the speaker. Sound then travels through the acoustic tubing to the user's ear, where the end of the acoustic tubing is held in place by the earpiece. In this manner, communications received by the two-way radio can be heard without letting others nearby hear the communications as well.

However, such earpieces do not typically block a substantial amount of loud ambient sound. Consequently, such earpieces do not provide adequate hearing protection. Indeed, some such earpieces are not intended to provide any hearing protection at all.

Although contemporary earplugs and earpieces are generally suitable for their intended purposes, they suffer from inherent deficiencies that tend to detract from their overall usefulness. It is therefore desirable to provide an earplug/earpiece that provides protection from noise exposure while being comfortable to wear for extended periods of time and while not tending to work its way loose or otherwise become less effective. It is also desirable to provide such earpieces that can be used with two-way radios and the like.

### BRIEF SUMMARY

Systems and methods for making and using an ear insert that protects the ear from ambient noise are disclosed. For example, an ear insert can comprise a stem that is configured to be inserted into an ear canal. The stem can be attached to an ear insert that is disposed within the conchae of the user's ear. One or more attachments can be configured to attach to the stem in a manner that at least partially defines a fit of the ear insert within an ear canal.

For example, the attachments can comprise one or more rubber toroids or o-rings that can be selected according to their size so as to provide a desirable fit of the insert within the ear canal. In this manner, the undesirable leakage of ambient noise into the ear canal is mitigated and hearing protection is enhanced.

An ear insert can comprise a stem configured to be inserted into an ear canal and means for mitigating transmission of sound through an ear canal, wherein the means is configured to attach to the stem in a manner that it at least partially defines a fit of the ear insert within an ear canal.

An earpiece assembly can comprise an earpiece configured to be disposed generally within the conchae of a user's ear and an ear insert attached to the earpiece. The ear insert can be configured to be disposed substantially within the user's ear canal. The earpiece can comprise a stem and at least one attachment configured to attach to the stem in a manner that at least partially defines a fit of the ear insert within an ear canal.

A method of fitting an ear insert can comprise attaching at least one attachment to a stem, wherein the stem is configured to be disposed within the ear canal. The attachment can contact tissue of the ear canal about a substantial portion of the periphery thereof in a manner that tends to seal the ear canal so as to inhibit the transmission of sound therethrough. Thus, the stem and the attachment(s) cooperate to block undesirable sound from reaching the eardrum and causing damage to the ear.

This invention will be more fully understood in conjunction with the following detailed description taken together with the following drawings.

### BRIEF DESCRIPTION OF THE DRAWINGS

FIG. 1 is a semi-schematic perspective view of an earpiece attached to an insert, wherein attachments, e.g., o-rings, can be added to the insert so as to vary the fit of the insert within the ear canal according to an exemplary embodiment of the present invention;

FIG. 2 is a semi-schematic perspective view of the earpiece, insert, and o-rings of FIG. 1, wherein the o-rings have been added to the insert and are disposed beneath flanges of the insert;

FIG. 3 is a semi-schematic perspective view of the insert of FIG. 1, providing exemplary dimensions thereof;

FIG. 4 is a semi-schematic front view of the upper portion of an example of a cable assembly, according to an example of an embodiment;

FIG. 5 is a semi-schematic front view of the lower portion of an example of a cable assembly, according to an example of an embodiment;

FIG. 6 is a semi-schematic view of a stereo cable assembly according to an example of an embodiment;

FIG. 7 is a semi-schematic top view of an o-ring, such as those of FIG. 1, providing exemplary dimensions thereof;

FIG. 8 is a semi-schematic cross-sectional side view taken along line 8 of FIG. 7;

FIG. 9 is a semi-schematic cross-sectional side view of an attachment having a non-circular minor cross-section;

FIG. 10 is a semi-schematic cross-sectional side view of an attachment having two flat sides; and

FIG. 11 is a semi-schematic cross-sectional side view of the attachment of FIG. 10 attached beneath the innermost flange of the insert of FIG. 1.

Embodiments of the present invention and their advantages are best understood by referring to the detailed description that follows. It should be appreciated that like reference numerals are used to identify like elements illustrated in one or more of the figures.

### DETAILED DESCRIPTION

According to an embodiment, an ear insert can have a variable fit so that the ear insert can be adjusted in a manner that accommodates the particular configuration of a user's ear canal. Thus, the ear insert can better seal the ear so as to inhibit the undesirable leakage of sound through the ear canal and can consequently mitigate hearing damage due to such sound.

More particularly, the size of the ear insert can be changed by adding and/or removing attachments. By changing the size of the ear insert, it can be made to better seal the ear while being comfortable to wear for extended periods of time. Further, a properly sized ear insert is less likely to loosen or fall out.

The ear insert can be part of an earplug or earpiece assembly that either mitigates all sound traveling though the ear canal or that selectively allows some sounds to travel through the ear canal while mitigating other sounds. The sounds that are allowed to travel through the ear canal can be from a device or can be ambient sounds, as discussed below.

The ear insert can comprise a stem that is configured to be inserted into a user's ear canal. One or more attachments can be configured to attach to the stem in a manner that at least partially defines a fit of the ear insert within an ear canal. Each attachment can individually vary the size, e.g., diameter, of the ear insert at a particular location along the length thereof so as to accommodate variations in the size, e.g., diameter of the ear canal of the user.

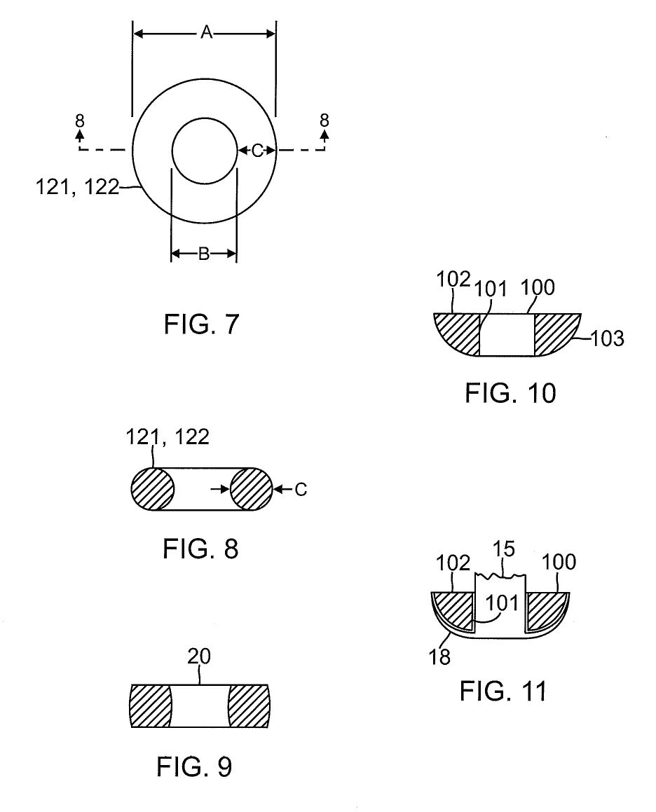

The attachments can have a minor cross-section that is either generally circular in configuration or is non-circular in configuration. Thus, the attachments can have a minor cross-section that is generally square, rectangular, or elliptical in configuration, for example. The attachments can have any desired minor cross-section. The minor cross-section of the insert can be defined herein as a cross-section that has a diameter of dimension C of FIGS. 7 and 8. Minor cross-sections are shown with cross-hatching in FIGS. 8 and 9.

The attachments can have a major cross-section that is either generally circular in configuration or is non-circular in configuration. The major cross-sectional configuration of the attachments can be matched to the cross-sectional configuration of the user's ear canal at the locations where the attachments are to be placed when the ear insert is worn. Any desired combination of major cross-sections can be provided on a single ear insert. For example, one attachment can have a circular major cross-section and another attachment can have a non-circular major cross-section. The major cross-section of the insert can be defined herein as a cross-section that that has a diameter of dimension A of FIG. 7. The major cross-section of an attachment will generally conform to the cross-section of the ear canal at the location where the attachment is disposed when the ear insert is worn.

The stem can have one or more flanges formed thereon. The attachments can be placed on the stem proximate the flanges. For example, the attachments can be placed upon the stem such that the attachments are substantially covered by the flanges. The flanges can curve outwardly (away from the eardrum) so as to substantially cover the attachments.

Alternatively, the flanges can not extend sufficiently far from the insert so as to substantially cover the attachments. For example, the flanges can comprise bumps upon the insert or rings around the insert that do not extend substantially far therefrom and that define detents that tend to hold the attachments in a desired position.

Any desired number of flanges can be used. For example, the stem can have one, two, three, four, five, or more flanges formed thereon. The flanges can be approximately evenly spaced upon the insert. Alternatively, the flanges can be grouped or otherwise spaced unevenly upon the insert. The flanges can be positioned at any desired location upon the insert.

Alternatively, the insert can have no flanges. In this instance, the attachments can be place in any desired position upon the stem. Whether or not the insert has flanges, the attachments can fit within grooves and/or indentations in the insert and/or can be adhesively bonded in place.

The ear insert can be configured to be attached to an earpiece. The earpiece can fit within the conchae of a user's ear. The earpiece can hold the ear insert in place and can help to define the position of the ear insert when it is worn. Thus, an earpiece assembly can comprise both an earpiece and an ear insert.

One or more embodiments comprise an earpiece with a flanged insert that is suitable for use as hearing protection, is suitable for facilitating listening to a two-way radio or the like, and is suitable for providing a combination of hearing protection and such listening. One or more embodiments can comprise a solid insert so as to substantially attenuate ambient sound. One or more embodiments can comprise a hollow or partially hollow insert so allow at least some ambient sound and/or sound from a device such as two-way radio, cellular telephone, or music player to pass therethrough.

The hollow insert can be defined by a bore extending at least partially though the insert. A filter, such as a Hochs filter, can be disposed within the bore so as to modify the spectrum of sound that passes through the insert. The filter can, for example, reduce the intensity of louder sounds. The filter can, for example, reduce the intensity of sounds outside of the voice range.

Thus, various combinations of sound attenuation and sound transmission can be provided. For example, a hollow or partially hollow insert can be configured so as to substantially attenuate some ambient sound (such as potentially harmful loud noise), while allowing some ambient sound (such as voices) to be heard.

According to one or more embodiments, an earpiece is held in place by anatomical structures of the ear and the earpiece holds the insert in place within the ear canal. That is, the earpiece prevents the insert from loosening or falling out of the ear canal.

According to one embodiment, the earpiece positions a sound port at the distal end of the insert near the eardrum, so that the volume of a two-way radio, for example, can be reduced. Thus, the earpieces determines how far into the ear the insert extends.

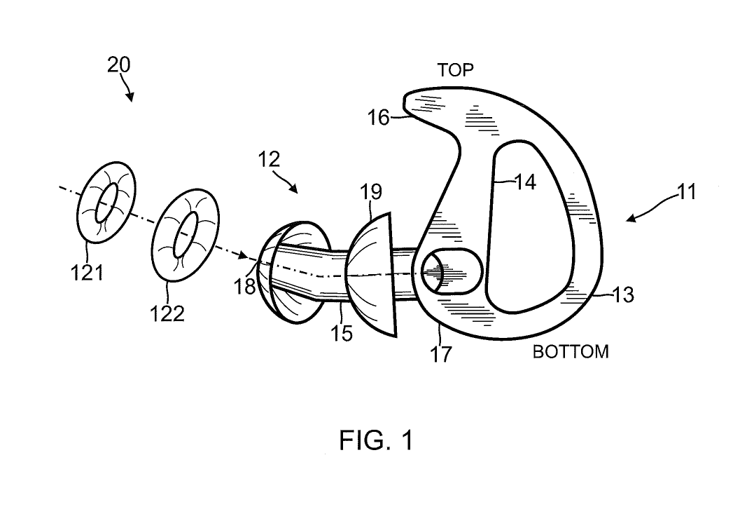

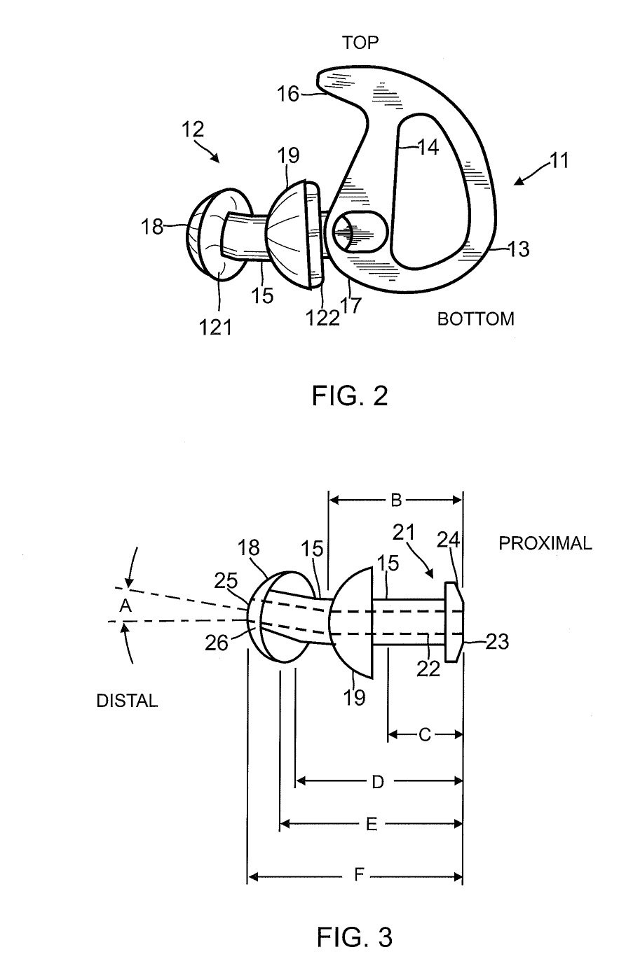

Referring now to FIGS. 1 and 2 an embodiment of the present invention comprises an earpiece 11 to which an ear insert 12 is attached. Earpiece 11 is configured to be disposed in the conchae of the outer ear. Insert 12 is configured to be disposed within the ear canal. The outer or major diameter of at least portions of the insert 12 can be varied so as to vary the fit of the insert 12 within the ear canal. The major diameter of the ear insert is the major diameter of an attachment thereof and is indicated as dimension A in FIG. 7.

For example, attachments 20 can be added to the insert 12 at desired locations along the length thereof so as to vary the major diameter of the insert 12 at these locations. In this manner, a desired fit of the insert 12 to the ear canal can be obtained at these locations. Often, a snug but comfortable fit is desired. The fit can be tight enough so as to substantially mitigate sound leakage around the insert without causing discomfort. Such a fit tends to cause the insert to maintain its desired position within the ear canal, with or without the use of the earpiece 11.

Thus, the ear insert 12 can be used either with or without the earpiece 11. When the insert 12 is used without the earpiece 11, it can extend far enough out of the ear so as to facilitate grasping and manipulation thereof. A small string or lanyard can extend from the proximal end of the insert 12 to facilitate such handling and manipulation.

Earpiece 11 can comprise a generally arcuate rib 13 that has upper and lower ends. Arcuate rib 13 can be attached to a straight rib 14 at the upper and lower ends of arcuate rib 13. An upper lobe 16 can be formed proximate where arcuate rib 13 and straight rib 14 join at the top of earpiece 11. Similarly, a lower lobe 17 can be formed proximate where arcuate rib 13 and straight rib 14 join at the bottom of earpiece 11.

Earpiece 11 can be configured to be disposed and held in place within the conchae of a human ear. More particularly, the crus and the antihelix of a user's ear can cooperate to capture upper lobe 16 while the tragus and antitragus cooperate to capture lower lobe 17. The antihelix and the antitragus can cooperate to capture arcuate rib 13. Thus, earpiece 11 can be configured to be captured by protrusions of the conchae. In this manner, earpiece 11 can be held firmly in place within the conchae and can therefore maintain insert 12 in a desired position within the ear canal.

Insert 12 can be either removably or permanently attached to earpiece 11. Insert 12 can be removably attached to earpiece 11 by friction fit, by detents, by threads, or by any other desired means. For example, insert 12 can be friction fit to earpiece 11 by sizing a proximal portion 21 (FIG. 3) of insert 12 so as fit tightly within an aperture of earpiece 11.

Insert 12 can be permanently attached to earpiece 11 by adhesive bonding, ultrasonic welding, or by any other desired means. Alternatively, insert 12 can be integrally formed to earpiece 11, such as by injection molding earpiece 11 and insert 12 within a common mold cavity. Thus, earpiece 11 and insert 12 can be formed either integrally or separately.

Earpiece 11 and insert 12 can be formed of a soft, resilient material to enhance comfort during use. Both earpiece 11 and insert 12 can be formed of the same material. For example, earpiece 11 and insert 12 can be formed of a resilient polymer, such as rubber or silicon rubber. Earpiece 11 and insert 12 can be formed of a material having a Shore A durometer of between 35 and 45, such as a Shore A durometer of approximately 40. Alternatively, the earpiece 11 and/or the insert 12 can be formed of a rigid material.

The attachments 20 can be either substantially resilient or substantially rigid. For example, the attachments 20 can be formed of a resilient polymer such as rubber or silicone rubber. The attachments 20 can have a Shore A durometer of between approximately 35 and approximately 45. For example, the attachments 20 can have a Shore A durometer of approximately 40.

Earpiece 11 can function as a stop to prevent insert 12 from being inserted too far into the ear. Earpiece 11 can also prevent insert 12 from being inadvertently removed or loosened from the ear. The length of insert 12 determines, at least in part, how close the tip thereof is positioned with respect to the eardrum.

According to one embodiment of the present invention, insert 12 comprises a stem 15 and two flanges, 18 and 19. Insert 12 can comprise any desired number of flanges, including no flanges at all. Stem 15 can either be solid (so as to substantially block sound) or hollow (so as to substantially transmit sound). Stem 15 can also be partially hollow (so as to selectively transmit sound).

With particular reference to FIG. 1, the attachments 20 can comprises o-rings 121 and 122. The o-rings 121, 122 of FIG. 1 are shown separated from the ear insert 12 and ready for attachment thereto. The o-rings 121, 122 can be attached to insert 12 by sliding the o-rings 121, 122 over the inner flange 18 and/or the outer flange 19. A readily removable lubricant, such as liquid soap, can facilitate such sliding.

With particular reference to FIG. 2, the o-rings 121, 122 can be disposed upon insert 12 such that the o-rings 121, 122 are next to and substantially covered by the flanges 18 and 19. Thus, the o-rings 121, 122 can maintain the flanges 18 and 19 in a generally fully opened or extended configuration and thereby mitigate collapse of the flanges.

Referring now to FIG. 3, the stem 15 can bend such that it angles upwardly to conform to the upward angle of the human ear canal. For example, stem 15 can bend such that it angles upwardly at an angle, Angle A, of approximately 30°. The distance between the proximal end of insert 12 and the distal end of outer flange 19, Dimension B, can be approximately 0.545 inch. The distance between the proximal end of insert 12 and the point where a filter (such as a Hoch filter) ends, Dimension C, can be approximately 0.304 inch. The distance between the proximal end of insert 12 and the bend in stem 15, Dimension D, can be approximately 0.680 inch. The distance between the proximal end of insert 12 and a distal end of inner flange 18, Dimension E, can be approximately 0.743 inch. The distance between the proximal end of insert 12 and the distal end of inner flange 18, Dimension F, can be approximately 0.870 inch.

The diameter of stem 15 can be approximately 0.189 inch. Stem 15 can optionally have a bore 22 formed therethrough. Bore 22 can have a diameter of approximately 0.094 inch. Outer flange 19 can have a radius of approximately 0.241 inch. Similarly, inner flange 18 can have a radius of approximately 0.193 inch. Thus, the radius of inner flange 18 can be substantially less than the radius of outer flange 19, so as to better accommodate the manner in which the human ear canal becomes narrower as it gets deeper.

The exemplary angle and dimensions discussed above provide a single insert that is suitable for use by a large number individuals. Those skilled in the art will appreciate that other dimensions are likewise suitable.

The distal end of bore 22 defines a sound output port 26. Insert 12 is configured such that sound output port 26 is positioned proximate the user's eardrum. That is, insert 12 can be configured so as to position a distal end 25 thereof proximate the eardrum. In this manner, sound transmitted through bore 22 (such as sound from a two-way radio), is brought close to the eardrum such that the volume of the sound required can be substantially reduced. For example, the insert can position sound output port 26 within one, two, or three millimeters of the eardrum. The insert can have a length of approximately ½ inch. The insert can have a length of ⅝ inch or more.

A head 23 can be formed upon the proximal end of stem 15 so as to enhance friction with respect to aperture 61 of earpiece 11 and/or so as to define detents that tend to keep insert 12 attached to earpiece 11. Bevels 24 can optionally be formed upon head 23 to better facilitate insertion of head 23 through aperture 61.

The insert can comprise a flanged insert, having flanges as shown in FIGS. 1-3. The flanges generally fill (close off) the ear canal and tend to block ambient sound. The insert can comprise any desired number flanges. For example, the insert can comprise three, four, five, or more flanges. The flanges can be generally circular, oval, or of any other desired shape. The flanges can be shaped so as to correspond to the shape of the user's ear canal at the position of the flange when worn. The flanges need not be identical, but rather can vary in size, shape, orientation and/or positions of attachment to the stem, for example.

The insert 12 can have a bore 22 formed therethrough to facilitate the transmission of sound from a speaker (such as via acoustic tubing connected to the speaker) to the user's eardrum. Alternatively, the insert can lack such a bore, so as to define an earplug, such as for attenuating ambient sound. Indeed, the bore and/or openings in the stem and/or flanges can be configured so as to selectively transmit and block desired sounds. Such selectivity can be based upon the frequency and/or intensity of the sound.

A user can wear one earpiece having an insert with a bore and one earpiece lacking a bore. The earpiece having an insert with a bore facilitates listening to a radio, while the earpiece having an insert without a bore at least partially blocks distracting and/or potentially harmful ambient sound.

One or more attachments 20 can be added the insert 12 to as to facilitate enhanced fit thereof with respect to the user's ear canal. The size, shape, and/or material of the attachments can be varied so as to provide the desired fit.

As discussed above, the two o-ring attachments 121 and 122 of FIG. 1 can be slid over the insert 12 so as to be positioned substantially under the flanges 18 and 19 as shown in FIGS. 1 and 2. The o-ring attachments can have different inner diameters (dimension B of FIG. 7) so as to fit upon portions of the insert 12 having different diameters. As shown in FIG. 1, o-ring 121 has slightly smaller inner and outer diameters than o-ring 122. The o-ring attachments 121 and 122 can have different outer diameters (dimension A of FIG. 7) so as to better fit the ear canal in portions thereof having different diameters.

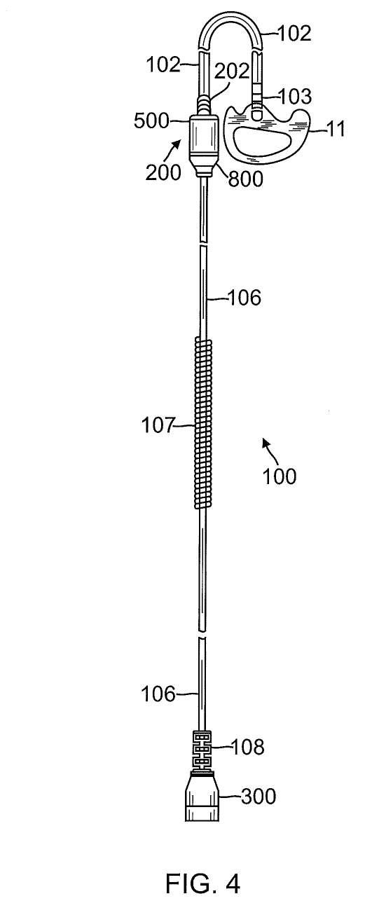

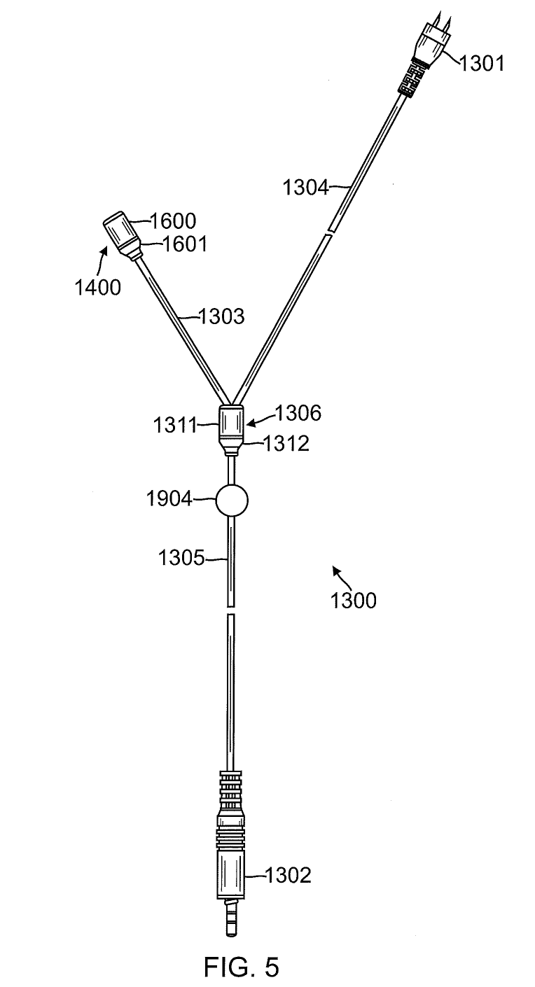

Referring now to FIG. 4, the ear insert 12 and/or earpiece 11 can be used with a cable assembly to facilitate communication, such as via a two-way radio or a cellular telephone. The ear insert 12 and/or earpiece 11 can similarly be used with a cable assembly to facilitate listening to music from an MP3® player, iPod®, or the like. The cable assembly can comprise an upper portion 100, as shown in FIG. 4 and a lower portion 1300, as shown in FIG. 5. Upper portion 100 can be connected to lower portion 1300 to form the complete cable assembly.

The upper portion 100 can comprise an earpiece 11 that is configured to fit within the conchae of a user's ear so as to transmit sound (such as incoming cellular telephone transmissions) to the user's eardrum. Examples of suitable earpieces are disclosed in U.S. patent application Ser. No. 11/411,314, filed on Apr. 26, 2006, and entitled EARPIECE WITH INSERT, the entire contents of which are hereby expressly incorporated by reference. The earpiece can be used either with or without the insert.

Earpiece 11 can be attached to acoustic tubing 102, such as via barbed fitting 103. Acoustic tubing 102 can be curved so as to facilitate easy routing thereof behind the ear. Speaker 200 can be worn directly behind the ear, behind the ear at the neck, in front of the ear, in the ear, or at any other desired location. Either acoustic tubing from speaker 200 or electrical cable to speaker 200 can pass by or behind the ear. Barbed fitting 103 can be an elbow fitting.

Acoustic tubing 102 can be attached to speaker 200, such as via a barbed fitting 202. Speaker 200 can comprise upper 500 and lower 800 housings. A multi-conductor electrical cable 106 can extend from speaker 200 to a connector, such as female connector 300 that electrically connects upper portion 100 of the cable assembly 2000 to lower portion 1300. For example, electrical cable 106 can comprise two conductors that facilitate operation of speaker 200. Strain relief 108 can be provided for electrical cable 106 at connector 300. Electrical cable 106 can comprise coils 107 that allow it to stretch as necessary to fit a particular individual.

Electrical cable 106 can provide electrical signals to speaker 800. Speaker 800 can convert such electrical signals into acoustic signals representative thereof (and generally representative of speech or music such as that received from a cellular telephone, music player, or the like).

Referring now to FIG. 5, lower cable assembly 1300 can comprise a microphone 1400 that is configured to attach to the user's clothing, such as proximate the user's mouth. For example, microphone 1400 can clip to the user's lapel. A connector, such as male connector 1301, can facilitate electrical connection of lower cable assembly 1300 to upper cable assembly 100 to define completer cable assembly 2000, as mentioned above. The microphone 1400 can be omitted for applications that do not require it, such as listening to a music player.

A connector, such as stereo phono plug 1302, can be used to connect lower cable assembly 1300 (and consequently complete cable assembly 2000) to a personal electronic device. Phono plug 1302 can plug directly into the personal electronic device.

Cable 1303 facilitates electrical connection between microphone 1400 and connector 1302. Similarly, cable 1304 facilitates electrical connection between upper portion of cable assembly 100 and connector 1302. Cable 1303 and cable 1304 can join at Y-joint 1306 to form single cable 1305. Y-joint 1306 can comprise a housing similar in construction to the speaker housing. Cable 1303 can be omitted for use with a music player, such as an MP3® player or iPod®.

More particularly, an upper Y-joint housing portion 1311 and a lower Y-joint housing portion 1312 can be formed of a durable, substantially rigid material. For example, upper Y-joint housing portion 1311 and lower Y-joint housing portion 1312 can be formed of a metal or alloy, such as a metal or alloy comprised of aluminum (anodized aluminum, for example), titanium, magnesium, or steel. Alternatively, upper Y-joint housing portion 1311 and lower portion 1312 can be formed of a polymer, such as ABS, polycarbonate, or high density polyethylene. Upper portion 1311 can attach to lower portion 1312 via threads, friction fit, adhesive bonding, ultrasonic welding, or by any other desired method.

Microphone 1400 can be removably attachable to lower cable assembly 1300, such as via a connector. Alternatively, microphone 1400 can be permanently attached to 1300 lower cable assembly, such as by being an integral part thereof.

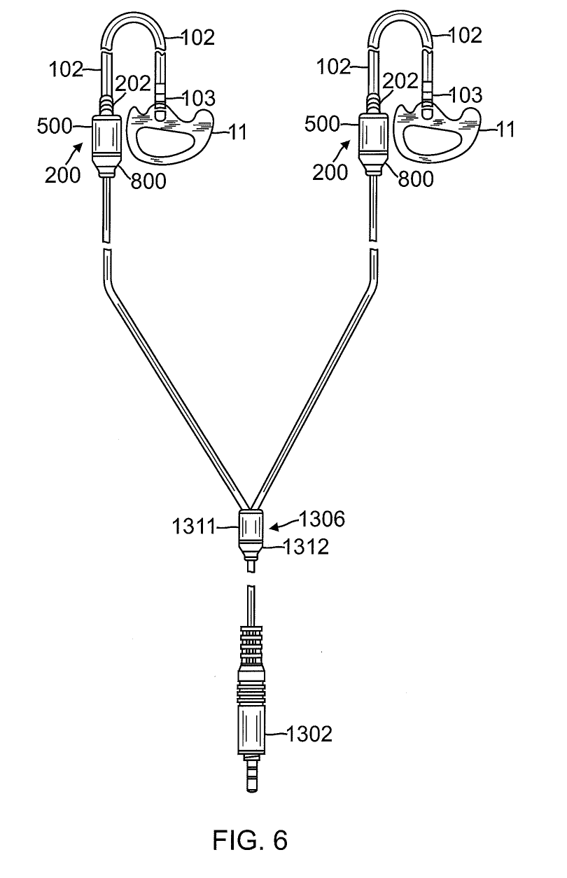

Referring now to FIG. 6, Y-joint 1306 can be used to form a two speaker/two earpiece cable, such as for stereo listening to a music player. Optionally, the microphone 1400 can be included, such as by additionally having microphone cable 1303 branch off from Y-joint 1306 as well.

Instead of having two separate speakers for a stereo configuration as shown in FIG. 6, the cable assembly can alternatively have a single speaker that provides sound to both earpieces to define a monaural configuration. For example, a single speaker can be placed at Y-joint 1306 and two acoustic tubes can lead therefrom to earpieces 11.

Various combinations of sound attenuation and sound transmission can be provided. For example, a hollow or partially hollow insert can be configured so as to substantially attenuate some ambient sound (such as potentially harmful loud noise), while allowing some ambient sound (such as voices) to be heard. Optionally, the insert can comprise one or more openings that allow a substantial portion of ambient sound to be heard, while also allowing communications, such as cellular telephone communications, to be heard. Optionally, a filter can be used to selectively allow sounds to be heard.

The cable assemblies of FIGS. 4-6 are disclosed in U.S. Ser. No. 11/696,987 filed on Apr. 5, 2007, and entitled CELLULAR TELEPHONE CABLE ASSEMBLY, the entire contents of which are hereby expressly incorporated by reference.

Referring now to FIGS. 7 and 8, the attachments 20 can be provided in a variety of sizes, i.e., outer diameters and/or cross-sectional configurations, so as to facilitate fitting of the insert to a variety of sizes and configuration of ear canals. For example, an attachment can be an o-ring 121, 122. The o-ring 121, 122 can have an outer diameter, dimension A, that is between approximately 4 mm and approximately 12 mm. The o-rings 121, 122 can have an inner diameter of between approximately 2 mm and approximately 4 mm. The o-rings 121, 122 can have a minor diameter of between approximately 1 mm and approximately 6 mm.

As used herein, the term “minor diameter” can be defined as the diameter of a cross-section of the material of the attachment or o-ring, as designated by dimension C in FIGS. 7 and 8.

Referring now to FIG. 9, the cross-sectional configuration of the attachment 20 can alternatively be non-circular. For example, the cross-sectional configuration of the attachment can be more rectangular or square. The attachments 20 can have any desired cross-sectional configuration.

Referring now to FIG. 10, an attachment 100 can have two substantially flat surfaces. For example, the surface 101 that contacts the stem 15 and the surface 100 that is opposite the flange 18 can be flat. The surface 103 that contacts the flange can be rounded so as to better conform to the shape of the flange 18. By making the surface 101 that contacts the stem 15 flat, the attachment is less likely to move undesirably upon the shaft 15. Making the surface 102 opposite the flange 18 flat provides a better fit of the attachment 100 to the insert 12.

Referring now to FIG. 11, the attachment 100 is shown installed upon the insert 12. Surface 101 encircles and generally contacts stem 15. Surface 102 is opposite flange 18.

A method of fitting an ear insert can comprise attaching at least one attachment to a stem. The attachments can be selected from a plurality of available attachments of various different sizes, shapes, and/or material. The sizes, shapes, and/or materials can be selected so as to compliment the size and shape of the user's ear canal. That is, larger attachments (those have larger outer diameters) can be selected for positions upon the stem corresponding to larger (larger inner diameter) portions of the user's ear canal. The attachments can be selected such that when the stem is disposed within the ear canal, then the attachment contacts tissue of the ear canal about a substantial portion of the periphery thereof.

Attachments can be selected during the fitting process by either the use of measurements or by trial and error. The ear canal can be measured, either quantitatively or qualitatively and the size of the attachment can be determined from such measurements. Either alternatively or in combination with such measurement, attachments can be tried and used or not used based upon their comfort and effectiveness.

One or more embodiments of the present invention can be used to mitigate exposure to ambient sound. For example, one or more embodiments can be used alone, e.g., without an electronic device. Alternatively, one or more embodiments can be used with an electronic device such as a two-way radio, a cellular telephone, a computer, a music player (such as an MP3® player or an iPod®), or the like. When used with an electronic device, the ear insert can still mitigate exposure to ambient sound.

One or more embodiments substantially mitigate the transmission of loud ambient sound through the ear canal so as to enhance hearing protection. One or more embodiments substantially block the transmission of sound around the stem of an ear insert by enhancing the fit of the ear insert in the ear canal.

Embodiments described above illustrate, but do not limit, the invention. It should also be understood that numerous modifications and variations are possible in accordance with the principles of the present invention. Accordingly, the scope of the invention is defined only by the following claims.

1. An ear insert comprising:
a stem configured to be inserted into an ear canal; and
at least one attachment configured to attach to the stem in a manner that at least partially defines a fit of the ear insert within the ear canal.

2. The ear insert as recited in claim 1, wherein the stem is substantially solid.

3. The ear insert as recited in claim 1, wherein at least one attachment is generally toroidal in configuration.

4. The ear insert as recited in claim 1, wherein at least one attachment comprises an o-ring.

5. The ear insert as recited in claim 1, wherein at least one attachment comprises an o-ring having an outer diameter of between approximately 4 mm and approximately 12 mm.

6. The ear insert as recited in claim 1, wherein at least one attachment comprises an o-ring having an inner diameter of between approximately 2 mm and approximately 4 mm.

7. The ear insert as recited in claim 1, wherein at least one attachment comprises an o-ring having a minor diameter of between approximately 1 mm and approximately 6 mm.

8. The ear insert as recited in claim 1, wherein at least one attachment comprises at least one o-ring having a minor cross-section that is generally circular in configuration.

9. The ear insert as recited in claim 1, wherein at least one attachment comprises at least one o-ring having a minor cross-section that is non-circular in configuration.

10. The ear insert as recited in claim 1, wherein at least one attachment is resilient.

11. The ear insert as recited in claim 1, wherein at least one attachment is comprised of rubber.

12. The ear insert as recited in claim 1, wherein at least one attachment has a Shore A durometer of between approximately 35 and approximately 45.

13. The ear insert as recited in claim 1, wherein at least one attachment has a Shore A durometer of approximately 40.

14. The ear insert as recited in claim 1, further comprising at least one flange formed upon the stem.

15. The ear insert as recited in claim 1, further comprising one flange formed upon the stem.

16. The ear insert as recited in claim 1, further comprising two flanges formed upon the stem.

17. The ear insert as recited in claim 1, further comprising three flanges formed upon the stem.

18. The ear insert as recited in claim 1, wherein the insert is configured to be attached to an earpiece.

19. The ear insert as recited in claim 1, wherein the insert is configured to be attached to an earpiece that is disposed substantially within a conchae of the ear.

20. The ear insert as recited in claim 1, wherein a surface of the attachment that contacts the stem is substantially flat in cross-section.

21. The ear insert as recited in claim 1, wherein a surface of the attachment that opposite a flange of the ear insert is substantially flat in cross-section.

22. An ear insert comprising:
a stem configured to be inserted into an ear canal; and
means for mitigating transmission of sound through an ear canal, the means being configured to attach to the stem in a manner that at least partially defines a fit of the ear insert within an ear canal.

23. An earpiece assembly comprising:
an earpiece configured to be disposed generally within a conchae;
an ear insert attached to the earpiece and configured to be disposed generally within an ear canal, the earpiece comprising:
a stem; and
at least one attachment configured to attach to the stem in a manner that at least partially defines a fit of the ear insert within an ear canal.

24. A method of fitting an ear insert, the method comprising attaching at least one attachment to a stem, wherein the stem is configured to be disposed within the ear canal and wherein the attachment contacts tissue of the ear canal about a substantial portion of the periphery thereof.
